(19) United States
(12) Patent Application Publication
(10) Pub. No.: US 2021/0321978 A1
(43) Pub. Date: Oct. 21, 2021

(54) FAT LAYER IDENTIFICATION WITH ULTRASOUND IMAGING

(71) Applicant: KONINKLIJKE PHILIPS N.V., Eindhoven (NL)

(72) Inventors: Man NGUYEN, Melrose, MA (US); Raghavendra SRINIVASA NAIDU, Auburndale, MA (US); Christine SWISHER, San Diego, CA (US); Hua XIE, Cambridge, MA (US)

(21) Appl. No.: 17/272,989

(22) PCT Filed: Aug. 30, 2019
(86) PCT No.: PCT/EP2019/073164

§ 371 Date: Mar. 3, 2021

Related U.S. Application Data
(60) Provisional application No. 62/727,276, filed on Sep. 5, 2018.

(51) Int. Cl. A61B 8/08; A61B 8/14; A61B 8/00; G06N 3/04

(52) U.S. Cl. CPC A61B 8/0858; A61B 8/14; A61B 8/463; G06N 3/04; A61B 8/5269; A61B 8/085; G06N 3/0454; A61B 8/469

(57) ABSTRACT

The present disclosure describes imaging systems configured to identify features within image frames and improve the frames by implementing image quality adjustments. An ultrasound imaging system can include a transducer configured to acquire echo signals responsive to ultrasound pulses transmitted toward a target. The system can also include a user interface configured to display an image and one or more processors configured to identify one or more features within the image. The processors can cause the interface to display elements associated with at least two image quality operations specific to the identified feature. A first image quality operation can include a manual adjustment of a transducer setting, and a second image quality operation can include an automatic adjustment of the identified feature derived from reference frames including the identified feature. The processors can receive a user selection of one or more elements and apply the operations to modify the image.

### TECHNICAL FIELD

The present disclosure pertains to ultrasound systems and methods for identifying features such as fat layers via ultrasound imaging and modifying images based on the identified features. Particular implementations involve systems configured to identify and remove a fat layer and associated image artifacts from an ultrasound image, thereby improving image quality.

### BACKGROUND

Ultrasound imaging can be challenging when scanning patients with moderate to thick fat layers, especially when scanning abdominal regions. The fat causes acoustic attenuation to increase, and because sound waves usually travel at different speeds through fat tissue relative to other soft tissues, ultrasound beams propagating through fat tissue often become defocused, an effect known as phase aberration. More particularly, ultrasound beam focusing is achieved by applying specific delays to each transducer element based on the time-of-flight of the acoustic pulse, e.g., the length of time it takes an ultrasound echo signal to travel from specific anatomical points to each transducer receive element, or the length of time it takes a transmitted ultrasound signal to arrive at certain anatomical points from the transducer. Beamforming based on inaccurate sound speed assumptions can lead to inaccurate distance-time relationship calculations, which can generate defocused ultrasound beams characterized by wide primary beams and high side lobes, for example. Such defocusing may occur frequently when imaging the abdominal fat layer, through which sound waves typically travel at only about 1450 m/s, which is significantly slower than most surrounding tissues, where the waves often travel at about 1540 m/s. Consequently, images of regions containing fat layers, such as the abdomen, commonly include unwanted artifacts and generally suffer from poor quality. Existing technologies aimed at correcting or minimizing image artifacts caused by fat are excessively complicated to implement and frequently ineffective. New ultrasound systems capable of reducing or eliminating the image degradation caused by fat layers are thus necessary to improve imaging and assessment of anatomical targets beneath layers of fat.

### SUMMARY

The present disclosure describes ultrasound systems and methods for identifying and locating at least one feature, such as a fat layer, within an ultrasound image. In some examples, the feature can be identified by implementing a neural network. Various measurements of the feature, such as the thickness of an identified fat layer, can also be determined, automatically or manually, and an indication of the same displayed on a graphical user interface. Systems can generate annotated ultrasound images in which the feature, e.g., fat layer, is labeled or highlighted for further assessment, thereby alerting a user to the feature and any associated aberrations. Systems can also generate and display at least one recommended manual adjustment of a transducer setting based on the feature identified, such that implementing the adjustment may remove aberrations or image artifacts caused by the feature. In some embodiments, a second neural network trained to automatically remove or modify the identified feature from ultrasound images can also be implemented. By removing the feature from a particular image, the second neural network can generate a revised image that lacks the feature and the associated image artifacts caused by the feature. The revised image, having enhanced quality relative to the original image, can then be displayed for analysis. The disclosed systems and methods are applicable to a broad range of imaging protocols, but may be especially advantageous when scanning anatomical regions high in fat content, such as the abdominal region, where image degradation caused by fat-induced artifacts may be the most severe. While example systems and methods are described with respect to fat layer identification and associated image modification, it should be understood that this disclosure is not limited to fat layer applications, and various anatomical features and/or image artifacts can be identified, modified, and/or removed in accordance with the principles disclosed herein.

In accordance with some examples of the present disclosure, an ultrasound imaging system may include an ultrasound transducer configured to acquire echo signals responsive to ultrasound pulses transmitted toward a target region, and a graphical user interface configured to display an ultrasound image from at least one image frame generated from the ultrasound echoes. The system can also include one or more processors in communication with the ultrasound transducer and the graphical user interface. The processors can be configured to identify one or more features within the image frame and cause the graphical user interface to display elements associated with at least two image quality operations specific to the identified feature. A first image quality operation can include a manual adjustment of a transducer setting, and a second image quality operation can include an automatic adjustment of the identified feature derived from reference frames including the identified feature. The processors may be further configured to receive a user selection of at least one of the elements displayed by the graphical user interface and apply the image quality operations corresponding to the user selection to modify the image frame.

In some examples, the second image quality operation can be dependent on the first image quality operation. In some embodiments, the one or more features can be identified by inputting the image frame into a first neural network trained with imaging data comprising reference features. In some examples, the one or more features can include a fat layer. In some embodiments, the graphical user interface can be configured to display an annotated image frame in which the one or more features are labeled. In some examples, the first neural network can include a convolutional network defined by a U-net or V-net architecture further configured to delineate a visceral fat layer and a subcutaneous fat layer within the image frame. In some embodiments, the processors can be configured to modify the image frame by inputting the image frame into a second neural network, the second neural network trained to output a revised image frame in which the identified feature is omitted for display on the graphical user interface. In some examples, the second neural network can include a generative adversarial network. In some embodiments, the processors can be further configured to remove noise from the image frame prior to identifying the one or more features. In some examples, the one or more processors can be further configured to determine a dimension of the fat layer. In some embodiments, the dimension can include a thickness of the fat layer at a location within the fat layer specified by a user via the graphical user interface. In some examples, the target region can include an abdominal region.

In accordance with some examples of the present disclosure, a method of ultrasound imaging can involve acquiring echo signals responsive to ultrasound pulses transmitted toward a target region, displaying an ultrasound image from at least one image frame generated from the ultrasound echoes, identifying one or more features within the image frame, and displaying elements associated with at least two image quality operations specific to the identified feature. A first image quality operation can include a manual adjustment of a transducer setting, and a second image quality operation can include an automatic adjustment of the identified feature derived from reference frames including the identified feature. The method can further involve receiving a user selection of at least one of the elements displayed, and applying the image quality operation corresponding to the user selection to modify the image frame.

In some examples, the second image quality operation can be dependent on the first image quality operation. In some embodiments, the one or more features can be identified by inputting the image frame into a first neural network trained with imaging data comprising reference features. In some examples, the one or more features can include a fat layer. In some embodiments, the method may further involve displaying an annotated image frame in which the one or more features are labeled. In some examples, the image frame can be modified by inputting the image frame into a second neural network, the second neural network trained to output a revised image frame in which the identified feature is omitted. In some embodiments, the method can also involve determining a dimension of the one or more features at an anatomical location specified by a user.

Any of the methods described herein, or steps thereof, may be embodied in non-transitory computer-readable medium comprising executable instructions, which when executed may cause a processor of a medical imaging system to perform the method or steps embodied herein.

### BRIEF DESCRIPTION OF THE DRAWINGS

FIG. 1 is an cross-sectional illustration of layered muscle, fat and skin tissue and a corresponding ultrasound image thereof.

FIG. 2 is a block diagram of an ultrasound system in accordance with principles of the present disclosure.

FIG. 3A is a block diagram of a U-net convolutional network configured for fat tissue segmentation in accordance with principles of the present disclosure.

FIG. 3B is a block diagram of a V-net convolutional network configured for fat tissue segmentation in accordance with principles of the present disclosure.

FIG. 4 is a graphical user interface implemented in accordance with principles of the present disclosure.

FIG. 5 is a block diagram of a neural network configured for fat layer image removal in accordance with principles of the present disclosure.

FIG. 6 is a block diagram of coordinated neural networks configured to identify and remove fat layers from ultrasound images in accordance with principles of the present disclosure.

FIG. 7 is a flow diagram of a method of ultrasound imaging performed in accordance with principles of the present disclosure

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

Systems disclosed herein can be configured to implement deep learning models to identify at least one layer of fat present in a target region. The fat layer can be indicated on a user interface, and a recommended solution for eliminating or reducing the image degradation caused by the fat may be generated and optionally displayed. Embodiments also include systems configured to improve ultrasound images by employing a deep learning model trained to remove fat layers and associated image artifacts from the images and generate new images lacking such features. The disclosed systems can improve B-mode image quality, especially when imaging regions high in fat, such as the abdominal region. The systems are not limited to B-mode imaging or abdominal imaging, and may be applied to imaging various anatomical features, e.g., liver, lungs and/or various extremities, as the systems can be utilized to correct images containing fat at any anatomical location of a patient. The systems can also be utilized for various quantitative imaging modalities, in addition to or instead of B-mode imaging, to improve the accuracy and/or efficiency thereof. For example, the disclosed systems may be implemented for shear wave elastography optimization, beam pattern adjustment for acoustic attenuation, and/or backscattering coefficient estimation.

An ultrasound system according to the present disclosure may utilize various neural networks, for example a deep neural network (DNN), a convolutional neural network (CNN), a recurrent neural network (RNN), a generative adversarial network (GAN), an autoencoder neural network, or the like, to identify a fat layer and optionally remove the fat layer in a newly generated image. In various examples, a first neural network may be trained using any of a variety of currently known or later developed learning techniques to obtain a neural network (e.g., a trained algorithm or hardware-based system of nodes) that is configured to analyze input data in the form of ultrasound image frames and determine the presence of at least one fat layer therein. A second neural network may be trained to revise input data in the form of ultrasound image frames or data containing or embodying fat layers and remove the layers therefrom. Image artifacts created by fat-induced phase aberration can also be selectively removed by the second neural network. Without the fat layers and associated artifacts, image quality can be significantly enhanced, which may be manifested in improved clarity and/or contrast.

An ultrasound system in accordance with principles of the present invention may include or be operatively coupled to an ultrasound transducer configured to transmit ultrasound pulses toward a medium, e.g., a human body or specific portions thereof, and generate echo signals responsive to the ultrasound pulses. The ultrasound system may include a beamformer configured to perform transmit and/or receive beamforming, and a display configured to display, in some examples, ultrasound images generated by the ultrasound imaging system. The ultrasound imaging system may include one or more processors and at least one neural network, which may be implemented in hardware and/or software components. Embodiments may include two or more neural networks, which may be communicatively coupled or integrated into one multi-layered network, such that the output of the first network serves as the input to the second network.

The neural network(s) implemented according to the present disclosure may be hardware- (e.g., neurons are represented by physical components) or software-based (e.g., neurons and pathways implemented in a software application), and can use a variety of topologies and learning algorithms for training the neural network to produce the desired output. For example, a software-based neural network may be implemented using a processor (e.g., single or multi-core CPU, a single GPU or GPU cluster, or multiple processors arranged for parallel processing) configured to execute instructions, which may be stored in computer readable medium, and which when executed cause the processor to perform a trained algorithm for identifying fat layers present within an ultrasound image and/or for generating new images lacking the identified fat layers. The ultrasound system may include a display or graphics processor, which is operable to arrange the ultrasound images and/or additional graphical information, which may include annotations, confidence metrics, user instructions, tissue information, patient information, indicators, and other graphical components, in a display window for display on a user interface of the ultrasound system. In some embodiments, the ultrasound images and associated measurements may be provided to a storage and/or memory device, such as a picture archiving and communication system (PACS) for reporting purposes or future training (e.g., to continue to enhance the performance of the neural network), especially the revised images generated by systems configured to remove fat layers and associated artifacts from fat-labeled images.

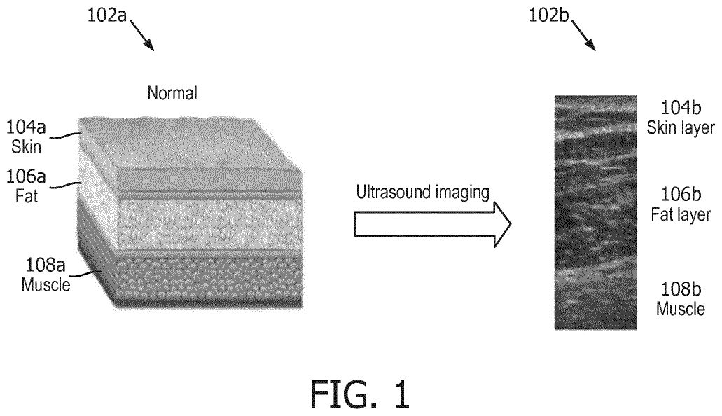

FIG. 1 shows a representation of a cross-section of normal tissue 102a, which includes an outer layer of skin 104a, a layer of fat 106a, and a layer of muscle 108a. Ultrasound imaging of the tissue can produce a corresponding image 102b of the skin layer 104b, the fat layer 106b, and the muscle layer 108b. As shown, each layer can appear distinct on the ultrasound image 102b, and the muscle layer 108b may appear brighter than the fat layer 106b. Existing techniques require users to identify and measure the fat layer 106b manually, and such techniques are not capable of removing the fat layer and associated artifacts from the image. Systems herein can identify one or more fat layers automatically and in some examples, process the corresponding images to improve image quality despite the existence of such fat layers. Systems herein may not be limited to the identification of fat layers, specifically, and may be configured to identify fat in any form, e.g., localized deposits, pockets or build-ups of various shapes. Example systems can also be configured to delineate visceral fat and subcutaneous fat. Subcutaneous fat can include an area about one centimeter above the umbilicus along the xipho-umbilical line. Subcutaneous fat layer thickness can be measured upon exhalation as the distance between the skin-fat interface and the outer edge of the linea alba. Visceral fat can be measured as the distance between the linea alba and the anterior aorta, about one centimeter above the umbilicus along the xipho-umbilical line.

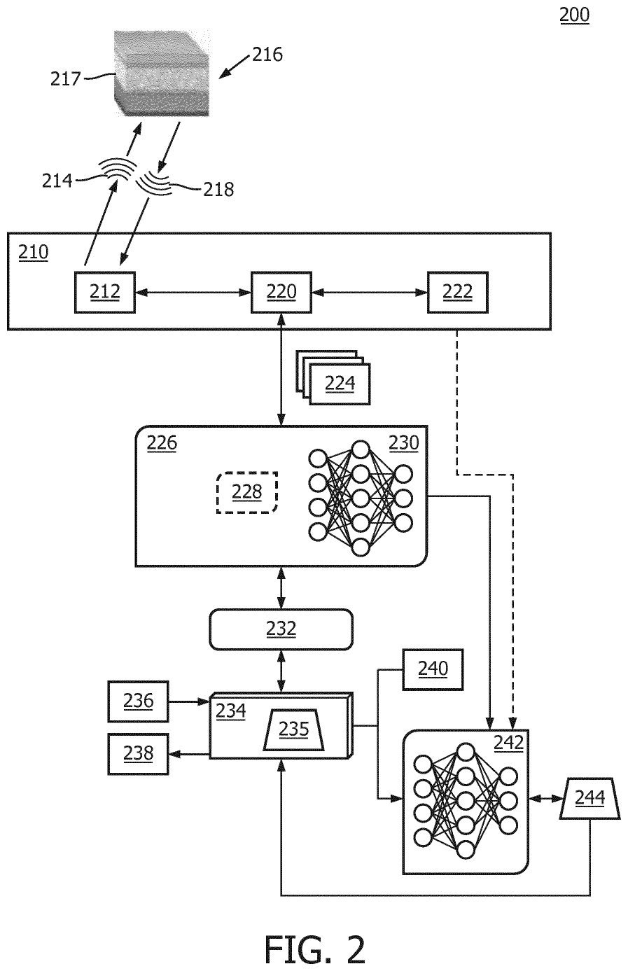

FIG. 2 shows an example ultrasound system according to principles of the present disclosure. The ultrasound system 200 may include an ultrasound data acquisition unit 210. The ultrasound data acquisition unit 210 can include an ultrasound probe which includes an ultrasound sensor array 212 configured to transmit ultrasound pulses 214 into a target region 216 of a subject, which may include an abdominal region, a chest region, one or more extremities and/or features thereof, and receive ultrasound echoes 218 responsive to the transmitted pulses. The region 216 may include a fat layer 217 of variable thickness. For example, the fat layer may range in thickness from about 0.1 to about 20 cm, about 1 to about 12 cm, about 2 to about 6 cm, or about 4 to about 5 cm. As further shown, the ultrasound data acquisition unit 210 can include a beamformer 220 and a signal processor 222, which can be configured to generate a stream of discrete ultrasound image frames 224 from the ultrasound echoes 218 received at the array 212. The image frames 224 can be communicated to a data processor 226, e.g., a computational module or circuitry, which may include a pre-processing module 228 in some examples, and may be configured to implement at least one neural network, such as neural network 230, trained to identify fat layer(s) within the image frames 224.

The ultrasound sensor array 212 may include at least one transducer array configured to transmit and receive ultrasonic energy. The settings of the ultrasound sensor array 212 can be preset for performing a particular scan, and can be adjustable during the scan. A variety of transducer arrays may be used, e.g., linear arrays, convex arrays, or phased arrays. The number and arrangement of transducer elements included in the sensor array 212 may vary in different examples. For instance, the ultrasound sensor array 212 may include a 1D or 2D array of transducer elements, corresponding to linear array and matrix array probes, respectively. The 2D matrix arrays may be configured to scan electronically in both the elevational and azimuth dimensions (via phased array beamforming) for 2D or 3D imaging. In addition to B-mode imaging, imaging modalities implemented according to the disclosures herein can also include shear-wave and/or Doppler, for example. A variety of users may handle and operate the ultrasound data acquisition unit 210 to perform the methods described herein.

The beamformer 220 coupled to the ultrasound sensor array 212 can comprise a microbeamformer or a combination of a microbeamformer and a main beamformer. The beamformer 220 may control the transmission of ultrasonic energy, for example by forming ultrasonic pulses into focused beams. The beamformer 220 may also be configured to control the reception of ultrasound signals such that discernable image data may be produced and processed with the aid of other system components. The role of the beamformer 220 may vary in different ultrasound probe varieties. In some embodiments, the beamformer 220 may comprise two separate beamformers: a transmit beamformer configured to receive and process pulsed sequences of ultrasonic energy for transmission into a subject, and a separate receive beamformer configured to amplify, delay and/or sum received ultrasound echo signals. In some embodiments, the beamformer 220 may include a microbeamformer operating on groups of sensor elements for both transmit and receive beamforming, coupled to a main beamformer which operates on the group inputs and outputs for both transmit and receive beamforming, respectively.

The signal processor 222 may be communicatively, operatively and/or physically coupled with the sensor array 212 and/or the beamformer 220. In the example shown in FIG. 2, the signal processor 222 is included as an integral component of the data acquisition unit 210, but in other examples, the signal processor 222 may be a separate component. In some examples, the signal processor may be housed together with the sensor array 212 or it may be physically separate from but communicatively (e.g., via a wired or wireless connection) coupled thereto. The signal processor 222 may be configured to receive unfiltered and disorganized ultrasound data embodying the ultrasound echoes 218 received at the sensor array 212. From this data, the signal processor 222 may continuously generate ultrasound image frames 224 as a user scans the target region 216. In some embodiments, ultrasound data received and processed by the data acquisition unit 210 can be utilized by one or more components of system 200 prior to generating ultrasound image frames therefrom. For example, as indicated by the dashed line and further described below, the ultrasound data can be communicated directly to the first or second neural network 230, 242, respectively, for processing before ultrasound image frames are generated and/or displayed.

The pre-processing module 228 can be configured to remove noise from the image frames 224 received at the data processor 226, thereby improving the signal-to-noise ratio of the image frames. The de-noising methods employed by the pre-processing module 228 may vary, and can include block-matching with 3D filtering in some examples. By improving the signal-to-noise ratio of ultrasound image frames, the pre-processing module 228 can improve the accuracy and efficiency of the neural network 230 when processing the frames.

In particular embodiments, neural network 230 may comprise a deep learning segmentation network configured to detect and optionally measure one or more fat layers based on one or more unique features of fat detected in the ultrasound image frames 224 or image data acquired by the data acquisition unit 210. In some examples, the network 230 can be configured to identify and segment a fat layer present within an image frame, and automatically determine the dimensions of the identified layer, e.g., thickness, length and/or width, at various locations, which may be specified by a user. The layer(s) can be masked or otherwise labeled on the processed images. In some examples, different configurations of the neural network 230 can segment fat layers present in 2D images or 3D images. Particular network architectures can include contracting and expanding cascades of convolutional and max pooling layers. Training the neural network 230 can involve inputting a large number of images containing annotated fat layers and images lacking fat layers, such that over time, the network learns to identify fat layers in non-annotated images in real time during an ultrasound scan.

The detected fat layer can be reported to a user via a display processor 232 coupled with a graphical user interface 234. The display processor 232 can be configured to generate ultrasound images 235 from the image frames 224, which can then be displayed in real time on the user interface 234 as an ultrasound scan is being performed. The user interface 234 may be configured to receive user input 236 at any time before, during or after an ultrasound procedure. In addition to the displayed ultrasound images 235, the user interface can be configured to generate one or more additional outputs 238, which can include an assortment of graphics displayed concurrently with, e.g., overlaid on, the ultrasound images 235. Such graphics may label certain anatomical features and measurements identified by the system, such as the presence and dimensions of at least one fat layer, e.g., visceral and/or subcutaneous, along with various organs, bones, tissues and/or tissue interfaces. In some examples, the fat layer(s) can be highlighted by outlining the contours of the fat and/or color-coding the fat areas. Fat thickness can be further calculated by determining the maximum, minimum and/or average vertical thickness of a masked fat area output from the segmentation network 230. In some embodiments, the outputs 238 can include selectable elements associated with image quality operations for improving the quality of a particular image 235. An image quality operation may include instructions for manually adjusting a transducer setting, e.g., adjusting the analog gain curve, applying preload to compress a detected fat layer, and/or turning on the harmonic imaging mode, in a manner that improves the image 235 by eliminating, reducing or minimizing one or more image artifacts or aberrations caused by the fat layer. The outputs 238 can include additional user-selectable elements and/or alerts to implement another image quality operation, which may depend on the first, embodying an automatic adjustment of the identified feature, e.g., fat layer, within the image 235 in a manner that eliminates, reduces or minimizes the feature and/or any associated artifacts or aberrations, as further described below. The graphical user interface 234 can then receive user input 236 to implement at least one of the quality operations, which can prompt the data processor 226 to modify the image frame(s) 224 containing the feature. In some examples, the user interface 234 can also receive image quality enhancement instructions differing from the instructions embodied in the outputs 238, for example instructions based on user knowledge and experience. Outputs 238 can also include annotations, confidence metrics, user instructions, tissue information, patient information, indicators, user notifications, and other graphic components.

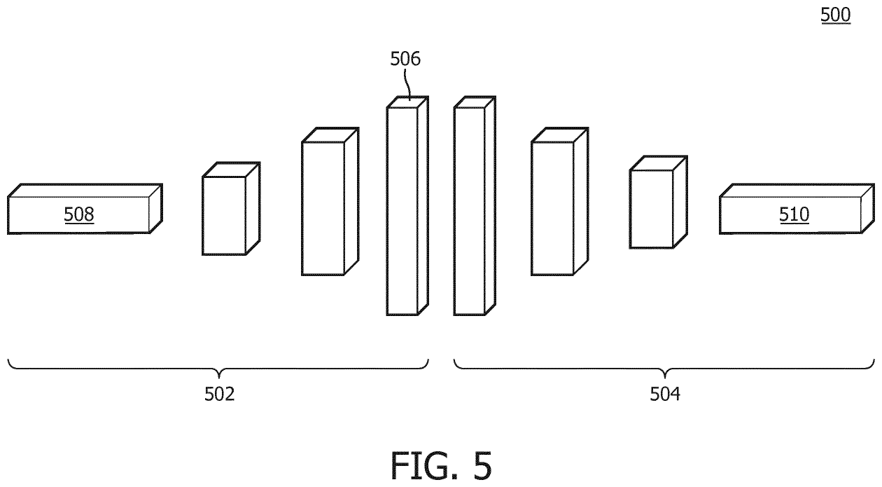

In some examples, the user interface 234 may be configured to receive a user instruction 240 specific to an automatic image quality operation. The user instruction 240 can be responsive to a selectable alert displayed on the user interface 234 or simply entered by the user. According to such examples, the user interface 234 may prompt the data processor 226 to automatically generate an improved image based on the determined presence of a fat layer by implementing a second neural network 242 configured to remove the fat layer(s) from the ultrasound image(s), thereby generating an improved image 244 lacking one or more fat layers and/or the image artifacts associated therewith. As shown in FIG. 2, the second neural network 242 can be communicatively coupled with the first neural network 230, such that the output of the first neural network, e.g., annotated ultrasound images in which the fat has been identified, may be input directly into the second neural network 242. In some examples, the second neural network 242 may include a Laplacian pyramid of adversarial networks configured to utilize a cascade of convolutional networks to generate images in a coarse-to-fine manner. Large-scale adjustments made to an input image containing at least one fat layer can be minimized to retain the most salient image characteristics while maximizing fine changes specific to the identified fat layers and associated image artifacts. The input received by the second neural network 242 can include ultrasound images containing fat layers, or image data embodying fat layers that has yet to be processed into full images. According to the latter example, the second neural network 242 can be configured to correct an image signal, e.g., by removing a fat layer and associated artifacts therefrom, in the channel domain of the ultrasound data acquisition unit 210. The architecture and mode of operation of the second neural network 242 may vary, as described below in connection with FIG. 5.

The configuration of the components shown in FIG. 2 may vary. For example, the system 200 can be portable or stationary. Various portable devices, e.g., laptops, tablets, smart phones, or the like, may be used to implement one or more functions of the system 200. In examples that incorporate such devices, the ultrasound sensor array may be connectable via a USB interface, for example. In some examples, various components shown in FIG. 2 may be combined. For instance, neural network 230 may be merged with neural network 242. According to such embodiments, the two networks may constitute sub-components of a larger, layered network, for example.

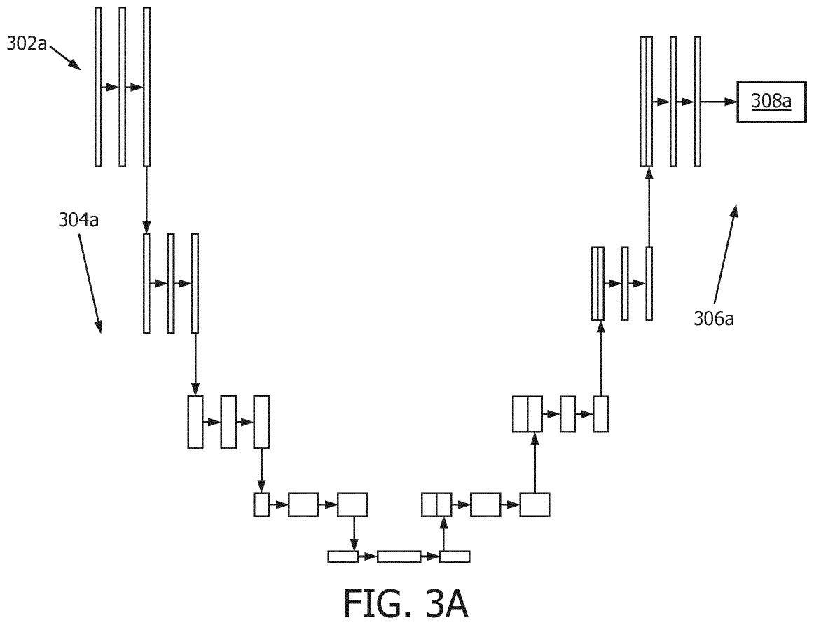

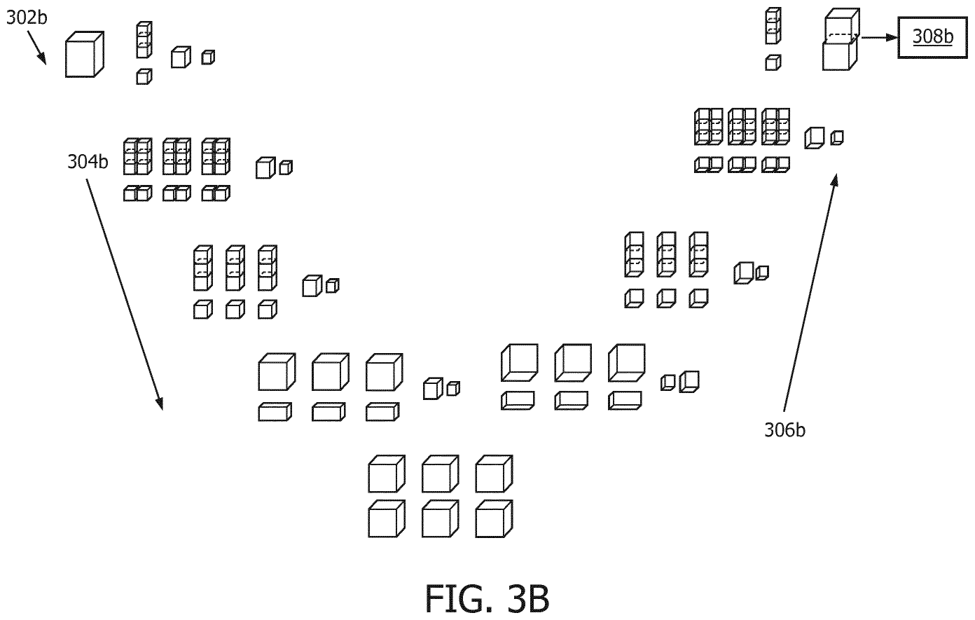

The particular architecture of network 230 can vary. In an example, the network 230 can comprise a convolutional neural network. In a specific example, the network 230 can comprise a convolutional auto-encoder with skip connections from encoder layers to decoder layers that are on the same architectural network level. For 2D ultrasound images, a U-net architecture 302a may be implemented in specific embodiments, as shown in the example of FIG. 3A. The U-net architecture 302a includes a contracting path 304a and an expansive path 306a. In one embodiment, the contracting path 304a can include a cascade of repeated 3×3 convolutions followed by a rectified linear unit and a 2×2 max pooling operation with downsampling at each step, for example as described by Ronneberger, O. et al. in “U-Net: Convolutional Networks for Biomedical Image Segmentation” (conditionally accepted at the Medical Image Computing and Computer Assisted Intervention Society, Published Nov. 18, 2015) (“Ronneberger”). The expansive path 306a can comprise sequential steps of up-convolution, each step halving the number of feature channels, as described by Ronneberger. The output 308a may include a segmentation map identifying one or more fat layers present within the initial image frame 224. The fat layer(s) or surrounding non-fat areas can be masked in some implementations, and in some examples, the output 308a may delineate non-fat areas, subcutaneous fat layers, and/or visceral fat layers with separate masking implemented for each tissue type. Training the network can involve inputting ultrasound images containing one or more fat layers and corresponding segmentation maps until the network learns to reliably identify fat layer(s) present within new images. Data augmentation measures may also implemented, as described by Ronneberger, to train the network when a small number of training images are available.

For 3D ultrasound images, a convolutional V-net architecture 302b may be implemented in specific embodiments, as shown in the example of FIG. 3B. The V-net architecture 302b can include a compression path 304b followed by a decompression path 306b. In one embodiment, each stage of the compression path 304b can operate at a different resolution and can include one to three convolutional layers performing convolutions on variously sized voxels, for example as described by Milletari, F. et al. in “V-Net: Fully Convolutional Neural Networks for Volumetric Medical Image Segmentation” (3D Vision (3DV), 2016 Fourth International Conference, 565-571, Published Oct. 25, 2016) (“Milletari”). In some examples, each stage can be configured to learn a residual function, which can achieve convergence in less time than preexisting network architectures, as further described by Milletari. The output 308b may include a three-dimensional segmentation map identifying one or more fat layers present within the initial image frame 224, which may include a delineation of non-fat, visceral fat, and/or subcutaneous fat. Training the network can involve end-to-end training by inputting three-dimensional images that include one or more fat layers and corresponding annotated images in which the fat layers are identified. Data augmentation measures may be implemented, as described by Milletari, to train the network when a limited number of training images, especially annotated images, are available.

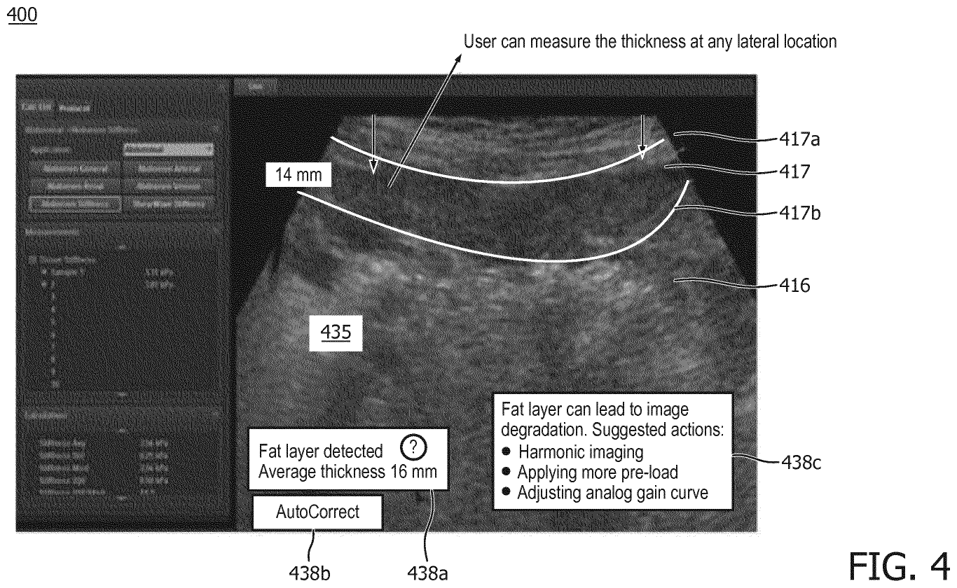

FIG. 4 shows an example of a graphical user interface 400 configured in accordance with the present disclosure. As shown, the interface 400 can be configured to show an ultrasound image 435 of a target region 416 that contains at least one fat layer 417, the boundaries of which are denoted by lines 417a and 417b. As further shown, the thickness of the fat layer 417 measures 14 mm at one location, which can be specified by a user, for example by interacting directly with the image 435 on a touch screen. Various example outputs are also illustrated, including a fat layer detection notification 438a, an “AutoCorrect” button 438b, and recommended instructions 438c for improving the quality of image 435 by adjusting system parameters. The fat layer detection notification 438a includes an indication of the average thickness of the fat layer 417, which in this particular example is 16 mm. By selecting the “AutoCorrect” button 438b, a user can initiate automatic removal of the fat layer 417 from the image via neural network generation of a revised image that retains all features of image 435 except the fat layer and any associated artifacts. Signal attenuation may also be reduced in the revised image. The recommended instructions 438c include instructions to initiate harmonic imaging, apply more pre-load, and adjust the analog gain curve. The instructions 438c can vary depending on the thickness and/or location of the fat layer detected in a given image, and/or the extent that a fat layer causes image artifacts to appear and/or generally degrades image quality. For instance, the instructions 438c may include recommended modifications to the position and/or orientation of the ultrasound probe used to acquire the image. In some implementations, the user interface 400 can display a revised image and selectable option to revert back to the original image, e.g., an “Undo Correction” button. According to such examples, a user can toggle back and forth between an image containing an annotated fat layer, and a new, revised image lacking the fat layer.

FIG. 5 shows an example of a neural network 500 configured to remove one or more fat layers and associated artifacts from an ultrasound image and generate a new, revised image lacking such features. This particular example comprises a generative adversarial network (GAN), but various network types can be implemented. The GAN 500 includes a generative network 502 and a competing discriminative network 504, for example as described by Reed, S. et al. in “Generative Adversarial Text to Image Synthesis” (Proceedings of the 33^{rd} International Conference on Machine Learning, New York, N.Y. (2016) JMLR: W&CP vol. 48). In operation, the generative network 502 can be configured to generate synthetic ultrasound image samples 506 lacking one or more fat layers and associated artifacts in feed-forward fashion based on input 508 comprised of text-labeled images, in which the identified fat layers are annotated. The discriminative network 504 can be configured to determine the likelihood that the samples 506 generated by the generative network 502 are real or fake, based in part on a plurality of training images containing fat layers and lacking fat layers. After training, the generative network 502 can learn to generate images lacking one or more fat layers from input images that contain one or more fat layers, such that the revised, nonfat images are substantially indecipherable from the actual ultrasound images but for the presence of fat and associated artifacts. In some examples, training network 500 may involve inputting pairs of controlled experimental images of phantom tissue both with and without a fat-layer near the surface. To generate a large number of sample images in a consistent manner such that each image has the same field of view in the phantom tissue, various robotic components and/or a motorized stage can be utilized, for example.

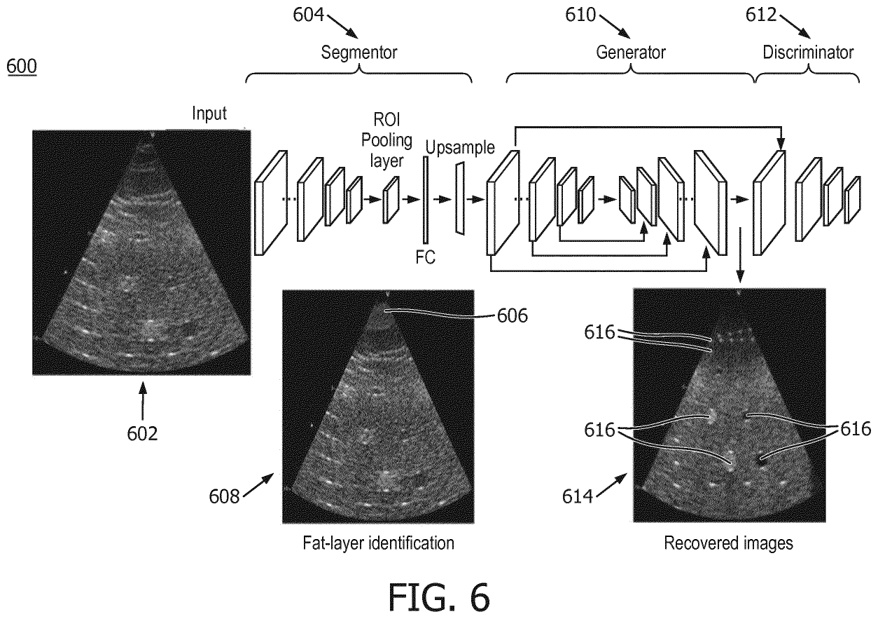

FIG. 6 shows coordinated system 600 of convolutional networks configured to identify and remove at least one fat layer from an original ultrasound image in accordance with principles of the present disclosure. An initial ultrasound image 602 can be input into a first convolutional network 604, which can be configured to segment and annotate a fat layer 606 present within the initial image, thereby generating an annotated image 608. The annotated image 608 can be input into a convolutional generator network 610 communicatively coupled with a convolutional discriminator network 612. As shown, the convolutional generator network 610 can be configured to generate a revised image 614 that lacks the fat layer 606 identified and labeled by the first convolutional network 604. Multiple anatomical features 616 appear more distinct in the revised image 614 due to the absence of fat layer 606 and the image degradation it causes. The organization of networks 604, 610 and 612 may vary in embodiments. One or more of images 602, 608 and/or 614 can be displayed for user analysis on a graphical user interface in various examples.

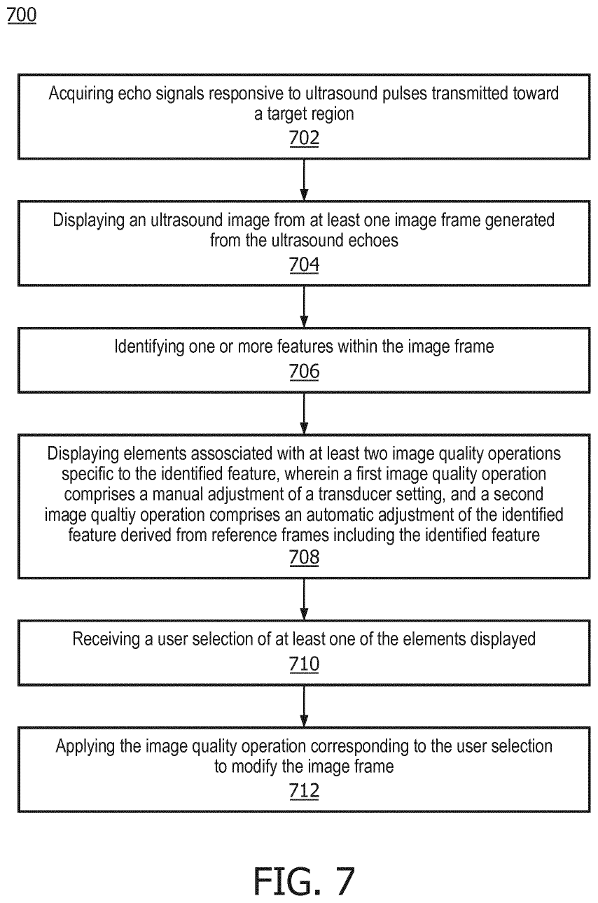

FIG. 7 is a flow diagram of a method of ultrasound imaging performed in accordance with principles of the present disclosure. The example method 700 shows the steps that may be utilized, in any sequence, by the systems and/or apparatuses described herein for identifying and optionally removing one or more fat layers from an ultrasound image, for example during an abdominal scan. The method 700 may be performed by an ultrasound imaging system, such as system 100, or other systems including, for example, a mobile system such as LUMIFY by Koninklijke Philips N. V. (“Philips”). Additional example systems may include SPARQ and/or EPIQ, also produced by Philips.

In the embodiment shown, the method 700 begins at block 702 by “acquiring echo signals responsive to ultrasound pulses transmitted toward a target region.”

The method continues at block 704 by “displaying an ultrasound image from at least one image frame generated from the ultrasound echoes.”

The method continues at block 706 by “identifying one or more features within the image frame.”

The method continues at block 708 by “displaying elements associated with at least two image quality operations specific to the identified feature, wherein a first image quality operation comprises a manual adjustment of a transducer setting, and a second image quality operation comprises an automatic adjustment of the identified feature derived from reference frames including the identified feature.”

The method continues at block 710 by “receiving a user selection of at least one of the elements displayed.”

The method continues at block 712 by “applying the image quality operation corresponding to the user selection to modify the image frame.”

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound imaging system comprising:
an ultrasound transducer configured to acquire echo signals responsive to ultrasound pulses transmitted toward a target region;
a graphical user interface configured to display an ultrasound image from at least one image frame generated from the ultrasound echoes; and
one or more processors in communication with the ultrasound transducer and the graphical user interface, the processors configured to:
identify one or more features within the image frame;
cause the graphical user interface to display elements associated with at least two image quality operations specific to the identified feature, wherein a first image quality operation comprises a manual adjustment of a transducer setting, and a second image quality operation comprises an automatic adjustment of the identified feature derived from reference frames including the identified feature;
receive a user selection of at least one of the elements displayed by the graphical user interface; and
apply the image quality operation corresponding to the user selection to modify the image frame.

2. The ultrasound system of claim 1, wherein the second image quality operation is dependent on the first image quality operation.

3. The ultrasound system of claim 1, wherein the one or more features are identified by inputting the image frame into a first neural network trained with imaging data comprising reference features.

4. The ultrasound system of claim 1, wherein the one or more features comprise a fat layer.

5. The ultrasound system of claim 1, wherein the graphical user interface is configured to display an annotated image frame in which the one or more features are labeled.

6. The ultrasound imaging system of claim 3, wherein the first neural network comprises a convolutional network defined by a U-net or V-net architecture further configured to delineate a visceral fat layer and a subcutaneous fat layer within the image frame.

7. The ultrasound imaging system of claim 1, wherein the processors are configured to modify the image frame by inputting the image frame into a second neural network, the second neural network trained to output a revised image frame, in which the identified feature is omitted, for display on the graphical user interface.

8. The ultrasound imaging system of claim 7, wherein the second neural network comprises a generative adversarial network.

9. The ultrasound imaging system of claim 1, wherein the one or more processors are further configured to remove noise from the image frame prior to identifying the one or more features.

10. The ultrasound imaging system of claim 4, wherein the one or more processors are further configured to determine a dimension of the fat layer.

11. The ultrasound imaging system of claim 10, wherein the dimension comprises a thickness of the fat layer at a location within the fat layer specified by a user via the graphical user interface.

12. The ultrasound imaging system of claim 1, wherein the target region comprises an abdominal region.

13. A method of ultrasound imaging, the method comprising:
acquiring echo signals responsive to ultrasound pulses transmitted toward a target region;
displaying an ultrasound image from at least one image frame generated from the ultrasound echoes;
identifying one or more features within the image frame;
displaying elements associated with at least two image quality operations specific to the identified feature, wherein a first image quality operation comprises a manual adjustment of a transducer setting, and a second image quality operation comprises an automatic adjustment of the identified feature derived from reference frames including the identified feature;
receiving a user selection of at least one of the elements displayed; and
applying the image quality operation corresponding to the user selection to modify the image frame.

14. The method of claim 13, wherein the second image quality operation is dependent on the first image quality operation.

15. The method of claim 13, wherein the one or more features are identified by inputting the image frame into a first neural network trained with imaging data comprising reference features.

16. The method of claim 13, wherein the one or more features comprise a fat layer.

17. The method of claim 13, further comprising displaying an annotated image frame in which the one or more features are labeled.

18. The method of claim 13, wherein the image frame is modified by inputting the image frame into a second neural network, the second neural network trained to output a revised image frame in which the identified feature is omitted.

19. The method of claim 13, further comprising determining a dimension of the one or more features at an anatomical location specified by a user.

20. A non-transitory computer-readable medium comprising executable instructions, which when executed cause a processor of a medical imaging system to perform the method of claim 13.
